(19) United States
(12) Patent Application Publication
(10) Pub. No.: US 2022/0378859 A1
(43) Pub. Date: Dec. 1, 2022

(54) METHOD FOR PREPARING BLOOD SUGAR CONTROL COMPOSITION CONTAINING CULTURED MUSHROOM MYCELIUM COMPLEX

(71) Applicant: GIUNCHAN CO., LTD., Cheonan-si, Chungcheongnam-do (KR)

(72) Inventors: Jong Yea PARK, Cheonan-si (KR); Mi Na PARK, Cheonan-si (KR); Hyun Min KIM, Asan-si (KR); Ya Ell KIM, Asan-si (KR)

(73) Assignee: GIUNCHAN CO., LTD., Cheonan-si, Chungcheongnam-do (KR)

(21) Appl. No.: 17/772,880

(22) PCT Filed: Dec. 3, 2019
(86) PCT No.: PCT/KR2019/016896

§ 371 Date: Apr. 28, 2022

(30) Foreign Application Priority Data
Oct. 29, 2019 (KR) 10-2019-0135631

(51) Int. Cl. A61K 36/07; A61K 36/074; A61P 3/10; A61K 9/16; A23L 33/10; A23P 10/20; A23L 31/00

(52) U.S. Cl. CPC A61K 36/07; A61K 36/074; A61P 3/10; A61K 9/16; A23L 33/10; A23P 10/20; A23L 31/00; A23V 2002/00

(57) ABSTRACT

Proposed is a method for complex culture of Innotus obliquus, Phellinus linteus, and Ganoderma lucidum. The cultured mycelium complex prepared by the above method has a high content of beta-glucan, which has excellent health functionality and can be used as additives or cooking seasonings for various foods. In addition, when raw meat is salted using the cultured mycelium complex, meat dishes with good taste and flavor can be easily prepared.

### TECHNICAL FIELD

The present disclosure relates to a method for preparing a blood sugar control composition containing a cultured mushroom mycelium complex.

### BACKGROUND ART

Phellinus linteus is also known as woody mud mushroom, and in Donguibogam, it is recorded in the Tangaek section under the name of Sangmok. Phellinus linteus grows naturally on the stems of mulberry trees and is yellow except for the pileus surface. In the early stage, Phellinus linteus looks like a lump of mud, but after they are fully grown, Phellinus linteus looks like a tongue sticking out of a tree stump, so Phellinus linteus is also called Suseol (tree's tongues). Since ancient times, Phellinus linteus was used for uterine bleeding and menstrual irregularities and has recently been reported to have excellent effects on tumor suppression, immunity enhancement, and skin-whitening.

Ganoderma lucidum occurs from the roots of broad-leaves trees in summer. Ganoderma lucidum is also known as a herb of the eternal youth of Qin Shi Huang, and in Boncho Gangmok, along with ginseng, this mushroom is placed in the ranks of good medicine. Ganoderma lucidum is known to be effective for respiratory diseases, nervous breakdowns, heart disease, and high blood pressure, lower cholesterol, and have anticancer effects because they have the effects such as cordial, antitussive, and small tumors.

On the other hand, in order to use the mushrooms as a mixed raw material for pharmaceuticals and functional foods, mushrooms, which are fruiting bodies, must be collected and used. However, there are problems that mushrooms themselves do not reproduce well in their natural state and that overuse of collection causes resource depletion and ecosystem destruction. A method of cultivating fruiting bodies using sawdust or the like is sometimes used, but this method also takes several months of cultivation. Cultivating fruiting bodies has not been able to meet the demand for mass production due to the difficulty of requiring a large number of production facilities and expenses to supply raw materials that can be used industrially. As such, although the mushrooms are effective as anticancer drugs or immunoenhancing agents, their supply is limited, and mass production and attribute production are not easy, so they are not widely used. Therefore, with respect to the mushrooms, research on a mass culture method capable of producing a mycelium exhibiting an effect equivalent to that of a fruiting body of a mushroom fungus has been actively conducted recently.

Korea Patent No. 10-922311 discloses a method of the complex culture of Innotus obliquus, Phellinus linteus, Ganoderma lucidum, Sparassis, and Cordyceps mycelium, Korea Patent No. 10-1358648 discloses a method of the complex culture method of Innotus obliquus, Phellinus linteus, and Ganoderma lucidum, and Korea Patent No. 10-1652035 discloses a method of culturing a mushroom mycelium complex of Innotus obliquus, Phellinus linteus, Ganoderma lucidum, and chrysanthemum mushroom. However, the cultured mycelium complex differs in its enzyme activity or beta-glucan content depending on the culture conditions. Even when food is processed using the cultured mycelium complex, different tastes and flavors can be produced.

Accordingly, the present inventors have completed the present disclosure by confirming that the cultured mycelium complex of Innotus obliquus, Phellinus linteus, and Ganoderma lucidum obtained through the complex culture method and the composition using the same have a function of controlling blood sugar.

### RELATED ART LITERATURE

### Patent Literature

(Patent Literature 1) Korea Patent No. 10-1923408 (Title of the disclosure: Complex culture method of Innotus obliquus, Phellinus linteus, and Ganoderma lucidum, Applicant: Kiunchan Co., Ltd., registration date: Nov. 23, 2018)

(Patent Literature 2) Korea Patent No. 10-1652035 (Title of the disclosure: Method for producing mushroom mycelium complex of Innotus obliquus, Phellinus linteus, and Sparassis, Applicant: Kiunchan Co., Ltd., registration date: Aug. 23, 2016)

(Patent Literature 3) Korea Patent No. 10-1358648 (Title of the disclosure: Complex culture method of Innotus obliquus, Phellinus linteus, and Ganoderma lucidum using mushroom extract, Applicant: Taebong Lee, date of registration: Jan. 28, 2014)

(Patent Literature 4) Korea Patent No. 10-1269410 (Title of the disclosure: Complex culture method of Innotus obliquus, Phellinus linteus, Ganoderma lucidum, Sparassis, and cordyceps using a mushroom medium, Applicant: Taebong Lee, date of registration: May 24, 2013)

(Patent Literature 5) Korea Patent No. 10-0922311 (Title of the disclosure: Method for double culturing of Innotus obliquus, Phellinus linteus, Ganoderma lucidum, Sparassis, and cordyceps to produce substances containing activated sugar-related compounds, Applicants: Taebong Lee, date of registration: Oct. 12, 2009)

### DISCLOSURE

### Technical Problem

An objective of the present disclosure is to provide a method for preparing a blood sugar control composition containing a cultured mushroom mycelium complex.

### Technical Solution

The present disclosure relates to a method for preparing a blood sugar control composition containing a cultured mushroom mycelium complex.

The mushroom mycelium may be cultured through the following steps, the steps including: (step 1) culturing the mycelium of each mushroom after inoculation of the fruiting body tissues of Innotus obliquus, Phellinus linteus, and Ganoderma lucidum into potato dextrose agar (PDA), respectively;

(step 2) inoculating the mycelium complex of each of the three types of Innotus obliquus, Phellinus linteus, and Ganoderma lucidum cultured in the first step into potato dextrose broth (PDB);

(step 3) culturing potato dextrose broth (PDB) inoculated with 3 types of mycelium for 4 to 6 weeks;

(step 4) inoculating the mycelium obtained through the culture of the third step in the rice barley medium; and

(step 5) obtaining a cultured mycelium complex by additionally culturing the mycelium inoculated in the rice barley medium for 4 to 7 weeks.

The mycelium culture in the third step is preferably performed at a relative humidity of 10% to 30% and 25° C. to 30° C. In addition, although stationary culture for the initial 1 to 2 weeks, it is preferable to stir for 1 to 5 minutes for 1 to 2 times a day, and then, it is good for culturing the mycelium by stirring at 50 to 150 rpm with shaking.

The rice barley medium of the fourth step may be obtained by dehydrating after immersing the rice barley in water for 4 to 8 hours, adding 0.5 to 2 parts by weight of calcium carbonate based on 100 parts by weight of dehydrated rice barley, and sterilizing at 120 to 125° C. for 30 minutes to 2 hours.

Culturing in the fifth step is preferably performed at a relative humidity of 40% to 60% and 25° C. to 30° C.

The blood sugar control composition can be provided as a pharmaceutical composition or healthy functional food for preventing, treating, or making better type 1 diabetes or type 2 diabetes.

The present disclosure also provides a granule composition containing heat-treated products of Cucumis melo conomon, sweet pumpkin, Capsosiphon fulvescens, and Ecklonia cava in the powder of mushroom mycelium prepared by the above method for controlling blood sugar and diet.

The Cucumis melo conomon, sweet pumpkin, Capsosiphon fulvescens, and Ecklonia cava are used as they are, and in particular, Cucumis melo conomon, sweet pumpkin uses only the pulp from which the seeds are removed.

The heat-treated product may be heat-treated at 120° C. to 130° C. for 1 to 5 hours by adding 200 to 400 parts by weight of water to a mixture of 50 to 150 parts by weight of a sweet pumpkin, 30 to 70 parts by weight of a Capsosiphon fulvescens, and 30 to 70 parts by weight of a Ecklonia cava based on 100 parts by weight other than Cucumis melo conomon.

The granular composition may be obtained by adding 80 to 120 parts by weight of the heat-treated product based on 100 parts by weight of the mushroom mycelium powder.

The granular composition may further include locust adult powder and silkworm pupa powder, and 30 to 70 parts by weight of locust adult powder, and 30 to 70 parts by weight of silkworm pupa powder may be added to the mushroom mycelium powder. The locust or silkworm pupa is preferably stir-fried or heated at a temperature of 120° C. to 200° C. for 10 to 20 minutes.

Accordingly, the granular composition can be provided as a pharmaceutical composition or healthy functional food for preventing, treating, or making better type 1 diabetes or type 2 diabetes that requires blood sugar control.

Hereinafter, the present disclosure will be described in detail.

Potato dextrose agar (PDA) used for culturing mushroom mycelium in the present disclosure may be prepared by sterilizing a potato dextrose agar mixture in which 3 g to 5 g of potato starch, 10 g to 30 g of dextrose, and 10 g to 30 g of agarose are included based on the total volume of 1 liter. At this time, water may be added so as to have a total volume of 1 liter as a residual amount. The sterilization is preferably performed at least at 120° C. to 125° C. for 15 to 20 minutes. The most preferred preparation condition of potato dextrose agar (PDA) may be that 4 g of potato starch, 20 g of dextrose, and 15 g of agarose are sterilized after adding water to a total volume of 1 liter.

In addition, potato dextrose broth (PDB) used for culturing the mushroom mycelium in the present disclosure may be prepared by adding more water instead of agarose under the preparation conditions of potato dextrose agar (PDA). The most preferable preparing conditions for potato dextrose broth (PDB) may be that 4 g of potato starch and 20 g of dextrose are sterilized after adding water to a total volume of 1 liter.

When culturing each mushroom fruiting body separately in the first step, it is preferable that each mushroom fruiting body piece is cultured at 25° C. to 30° C. for 1 to 3 weeks after inoculation with a potato dextrose agar (PDA), and the cultivation period can be appropriately adjusted within 1 to 3 weeks according to the growing ability of the mushroom fruiting body. In addition, it is preferable that the relative humidity during culture in this condition is 10% to 60%. Even if the humidity is less than 10% or exceeds 60%, the growth of the mycelium may be slow.

However, if the culture period is less than one week, the mycelium may not grow well enough to activate subsequent liquid culture, and even if it exceeds three weeks, mycelium culture may not be well developed during subsequent liquid culture. In addition, even if the culture temperature is less than 25° C. or more than 30° C., it also affects the culture of mycelium during liquid culture, which is not desirable.

In the second step, when the three types of mycelium are complexly inoculated to potato dextrose broth (PDB), and when the mycelium of each mushroom cultured in the first step is complexly inoculated, the mycelium is inoculated in a certain amount and in a similar amount. However, more preferably, each cultured mycelium is cut into 0.5 mm² to 2 mm², and 3 to 7 sections for each mushroom mycelium may be inoculated with three types of mycelium complex in potato dextrose broth (PDB).

The mycelium culture of the third step is preferably performed at 25° C. to 30° C., in particular, when the culture temperature is less than 25° C., the mycelium may not grow well, and even if it exceeds 30° C., the culture of the mycelium is also may be slow. In addition, although stationary culture for the initial 1 to 2 weeks, it is preferable to stir for 1 to 5 minutes for 1 to 2 times a day, and then, it is good for culturing the mycelium by stirring at 50 to 150 rpm with shaking. On the other hand, the relative humidity during culture under this condition is not particularly limited, but it is preferable that the relative humidity is 10% to 30%.

Based on 1 kg of rice barley medium in the fourth step, the mycelium obtained through the culturing in the third step may be inoculated by 1 ml to 10 ml each as a culture medium. At this time, if the mycelium culture medium is inoculated with less than 1 ml, additional culture in the rice barley medium may not work well, and even if the culture medium is inoculated with more than 10 ml, the culture is not activated further, which is not preferable.

The rice barley medium of the fourth step may be obtained by dehydrating after immersing the rice barley in water for 4 to 8 hours, adding 0.5 to 2 parts by weight of calcium carbonate based on 100 parts by weight of dehydrated rice barley, and sterilizing at 120° C. to 125° C. for 30 minutes to 2 hours. At this time, if rice barley that is not sufficiently soaked in water and immersed for less than 4 hours is used, the moisture during mycelium culture may be insufficient, and thus the rice barley may not be used well as a nutrient source. Soaking in water for more than 8 hours is not preferable because it has the effect of delaying only the manufacturing time because more moisture is not absorbed into the rice barley.

Culturing in the fifth step is preferably performed at 25° C. to 30° C. If the culture temperature is less than 25° C., the mycelium may not grow well, and even if it exceeds 30° C., the culture of the mycelium may also be slow. In addition, it is preferable that the relative humidity during culture under this condition is 40% to 60%. Even if the humidity is less than 40% or exceeds 60%, the growth of the mycelium may be slow.

The present disclosure may provide a pharmaceutical composition including a cultured mycelium complex of Innotus obliquus, Phellinus linteus, and Ganoderma lucidum and a pharmaceutical excipient.

Each of the pharmaceutical compositions may be formulated and used in the form of an oral formulation such as powders, granules, tablets, capsules, suspensions, emulsions, syrups, aerosols, etc., external preparations, suppositories, and sterile injection solutions according to conventional methods. Carriers, excipients, and diluents that may be included in the pharmaceutical composition may include lactose, dextrose, sucrose, sorbitol, mannitol, xylitol, erythritol, maltitol, starch, acacia gum, alginate, gelatin, calcium phosphate, calcium silicate, cellulose, methylcellulose, microcrystalline cellulose, polyvinyl pyrrolidone, water, methyl hydroxybenzoate, propyl hydroxybenzoate, talc, magnesium stearate, and mineral oil. In the case of formulation, it is prepared using commonly used diluents or excipients such as fillers, extenders, binders, wetting agents, disintegrants, and surfactants. Solid preparations for oral administration include tablets, pills, powders, granules, capsules, etc., and these solid preparations are prepared by mixing include at least one excipient in the cultured mycelium complex of the present disclosure, such as starch, calcium carbonate, sucrose or lactose, gelatin, etc. In addition to simple excipients, lubricants such as magnesium stearate and talc are also used. Liquid formulations for oral administration include suspensions, solutions, emulsions, syrups, etc. In addition to water and liquid paraffin, which are commonly used simple diluents, various excipients such as wetting agents, sweeteners, air fresheners, preservatives, etc., may be included. Formulations for parenteral administration include sterile aqueous solutions, non-aqueous solutions, suspensions, emulsions, freeze-dried preparations, and suppositories. Vegetable oils such as propylene glycol, polyethylene glycol, and olive oil, injectable esters such as ethyl oleate, and the like may be used as the non-aqueous solvent and the suspension. Witepsol, Macrogol, Twin 61, cacao oil, laurin oil, glycero gelatin, etc., may be used as the suppository.

The dosage of the pharmaceutical composition of the present disclosure will vary depending on the age, sex, and weight of the subject to be treated, the specific disease or pathological condition to be treated, the severity of the disease or pathological condition, the route of administration, and by the judgment of the prescriber. Dosage determination based on these factors is within the level of one of ordinary skilled in the art, and generally, dosages range from 0.01 mg/kg/day to approximately 2000 mg/kg/day. A more preferred dosage is 1 mg/kg/day to 500 mg/kg/day. Administration may be administered once a day or may be administered in several divided doses. The above dosage does not limit the scope of the present disclosure in any way.

The pharmaceutical composition of the present disclosure may be administered to mammals such as mice, livestock, and humans by various routes. All methods of administration may be expected, such as oral, rectal or intravenous, intramuscular, subcutaneous, intrauterine dural, or cerebrovascular injection. Since the cultured mycelium complex of the present disclosure has almost no toxicity and side effects, it is a drug that can be safely used even when taken for a long period of time for the purpose of prevention.

In addition, the present disclosure provides a healthy functional food for regulating blood sugar, including a cultured mycelium complex of Innotus obliquus, Phellinus linteus, and Ganoderma lucidum, and a nutritively acceptable food supplement additive. The cultured mycelium complex of the Innotus obliquus, Phellinus linteus, and Ganoderma lucidum may be added to the health functional food of the present disclosure in an amount of 0.001% to 100% by weight. The healthy functional food of the present disclosure includes the form of tablets, capsules, pills, or liquids, and the food to which the cultured mycelium complex of the present disclosure may be added includes, such as various drinks, meat, sausage, bread, candies, snacks, noodles, ice cream, dairy products, soups, ionized beverages, beverages, alcoholic beverages, gum, tea, and vitamin complexes.

### Advantageous Effects

The present disclosure relates to a method for preparing a blood sugar control composition containing mushroom mycelium. The present disclosure may provide a mushroom complex mycelium with the best blood sugar control ability by a single culture of mushroom mycelium in potato dextrose agar (PDA), complex culture of mycelium in Potato dextrose broth (PDB), and culturing of cultured mycelium complex obtained therefrom in a barley medium.

### BEST MODE

Hereinafter, preferred embodiments of the present disclosure will be described in detail. However, the present disclosure is not limited to the embodiments described herein and may be embodied in other forms. Rather, it is provided so that this disclosure will be thorough and complete and will fully convey the spirit of the disclosure to those skilled in the art.

### Preparation Example 1. Complex Culture of Innotus obliquus, Phellinus linteus, and Ganoderma lucidum

The fruiting bodies of Innotus obliquus, Phellinus linteus, and Ganoderma lucidum were isolated and inoculated into PDA, and then the mycelium of each mushroom was cultured at 27° C. to 29° C. for 2 weeks. After preparing the PDB medium subdivided into 100 ml units, each mushroom mycelium cultured in PDA was cut into 1 mm² using a scalpel. The three strains were cut per one Erlenmeyer flask containing PDB medium were combined with 5 slices each.

A complexly inoculated medium was incubated in a stationary phase in a bio-oxygen demand incubator (BOD incubator, low-temperature incubator) for 1 week at 27° C. to 28° C. and 20% humidity, but was stirred for about 1 minute every day during incubation. After one week, the flask under the complex inoculation and culture was transferred to a shaking incubator and cultured at 27° C. and 100 rpm for 4 weeks to prepare a cultured mycelium complex culture solution.

After immersing rice barley for 6 hours, dehydrated for 8 hours, added 1 g of calcium carbonate based on 100 g of dehydrated rice barley, mixed evenly, and sterilized at 121° C. for 1 hour in an autoclave to prepare a rice barley medium. After completion of the sterilization, 5 ml of the cultured mycelium complex culture medium cultured for 5 weeks per 1 kg of the rice barley medium cooled to 25° C. was dispensed and inoculated. After inoculation, the cultured mycelium complex was cultured for 30 days in a culture room maintained at a temperature of 26° C. to 28° C. and a humidity of 45% to 50%.

The cultured mycelium complex was dried at 57° C. to 60° C. for 24 hours using a dryer while containing rice barley and pulverized using a pin mill grinder to prepare a powder.

Meanwhile, this method is the same as the method disclosed in Example 1 of Korea Patent No. 10-1923408.

### Comparative Preparation Example 1. Preparation of Each Mushroom Mycelium

Using the method of Example 1, but as a comparative condition, the single mycelium culture of Innotus obliquus, Phellinus linteus, and Ganoderma lucidum and the complex culture of Phellinus linteus and Innotus obliquus were performed in the same process to obtain a mushroom mycelium.

TABLE 1

| | Mycelium |
|---|---|
| Comparative Preparation | Innotus obliquus mycelium |
| Example 1-1 | |
| Comparative Preparation | Ganoderma lucidum mycelium |
| Example 1-2 | |
| Comparative Preparation | Phellinus linteus mycelium |
| Example 1-3 | |
| Comparative Preparation | Complex mycelium of Innotus |
| Example 1-4 | obliquus and Phellinus linteus |

### Experimental Example 1. Check the Blood Sugar Control Effect

### Experimental Example 1-1. Blood Sugar Control Effect in Animal Model of Type 1 Diabetes

The animal model of type 1 diabetes animal model was induced by intravenous injection of 50 mg/kg of alloxan (Alloxan, Sigma, MA, USA) to ICR mice (male, 7 weeks old, 7 animals per group). In the experimental group, the mushroom mycelium was orally administered for 3 days before the administration of alloxan once a day at 2 mg/mouse by suspension of dry powder in phosphate-buffered saline (PBS). In addition, blood was collected up to the 9th day at 3-day intervals after alloxan administration, and the blood glucose content was measured and shown in Table 2 below.

TABLE 2

| | Blood glucose (mg/dL) | | | | | | |
|---|---|---|---|---|---|---|---|
| | | | | Alloxan + | Alloxan + | Alloxan + | Alloxan + |
| | | | | Comparative | Comparative | Comparative | Comparative |
| | | Positive | Alloxan + | Preparation | Preparation | Preparation | Preparation |
| | | control | Example | Example | Example | Example | Example |
| | | Group | 1 | 1-1 | 1-2 | 1-3 | 1-4 |
| | Alloxan | (alloxan | mycelium | mycelium | mycelium | mycelium | mycelium |
| | Untreated | alone | administration | administration | administration | administration | administration |
| Condition | group | group) | group | group | group | group | group |
| 3 days | 99 | 496 | 125 | 434 | 421 | 421 | 425 |
| 6 days | 100 | 562 | 112 | 352 | 352 | 312 | 321 |
| 9 days | 98 | 582 | 105 | 312 | 314 | 321 | 294 |

Referring to Table 2, it can be confirmed that blood sugar levels are maintained almost similar to those of non-diabetic induction mice, an alloxan-free group, throughout the administration period in the animals of the cultured mycelium complex administration group.

### Experimental Example 1-2. Blood Sugar Control Effect in Animal Model of Type 2 Diabetes

The animal model of type 1 diabetes was used by purchasing db/db diabetic mice (5 weeks old, male). In the experimental group, the mushroom mycelium was suspended in PBS with dry powder and orally administered at 2 mg/mouse once a day for 8 weeks, and then the blood glucose content was measured and shown in Table 3 below.

TABLE 3

| | Blood glucose (mg/dL) | | | | | |
|---|---|---|---|---|---|---|
| | | | Comparative | Comparative | Comparative | Comparative |
| | | Preparation | Preparation | Preparation | Preparation | Preparation |
| | | Example | Example | Example | Example | Example |
| | | 1 | 1-1 | 1-2 | 1-3 | 1-4 |
| | | mycelium | mycelium | mycelium | mycelium | mycelium |
| | Untreated | administration | administration | administration | administration | administration |
| Condition | group | group | group | group | group | group |
| 8 week | 412 | 251 | 381 | 372 | 353 | 298 |

The db/db diabetic mouse is characterized by maintaining the blood glucose concentration at 390 to 420 mg/dL, but the results in Table 3 show that the cultured mycelium complex administration group in Preparation Example 1 is reduced by about 50% compared to the untreated group.

Through this animal experiment, it was confirmed that the cultured mycelium complex prepared in the present disclosure had very good blood sugar control ability.

Next, a formulation experiment was conducted to prepare a mushroom processed product in a state suitable for diabetic patients to eat and carry the cultured mycelium complex directly.

At this time, for the formulation, it was considered whether it is possible to control blood sugar quickly while easily relieving hunger during intake, whether it is possible to supply protein that may be insufficient for diabetic patients with insufficient muscle strength, and whether there is a good taste and aroma to continue taking the formulation.

In addition, the final form was decided to be prepared in the form of granules that are easy to take without water while being well packaged in disposable packaging paper.

Through preliminary experiments, in the powder mixture state, the powder mixture form was difficult to take it without water, so the powder mixture form was avoided. Since the hard pill formulation takes time to digest in the stomach after taking pill, fast blood sugar control is not good, so the pill formulation was also excluded from the experiment.

### <Preparation Example 2. Manufacture of Heat-Treated Products of Cucumis melo Conomon, Sweet Pumpkin, Capsosiphon Fulvescens, and Ecklonia cava>

Cucumis melo conomon fruit, sweet pumpkin fruit were purchased and Cucumis melo conomon, sweet pumpkin uses only the pulp from which the seeds are removed. Capsosiphon fulvescens, and Ecklonia cava in their raw state were purchased and used.

Using this prepared material, Cucumis melo conomon, sweet pumpkin, Capsosiphon fulvescens, and Ecklonia cava, and water was put in an autoclave as shown in Table 2 and heated at 121° C., 1.5 atm for 1 hour, and then cooled to room temperature. Next, the cooled contents were put into a food grinder and pulverized to prepare a heat-treated product in the form of porridge.

TABLE 4

| | Cucumis | | | | |
|---|---|---|---|---|---|
| | melo | Sweet | Capsosiphon | | |
| | conomon | Pumpkin | fulvescens | Ecklonia | Water |
| Condition | (g) | (g) | (g) | cava (g) | (g) |
| Preparation | 100 | 100 | 50 | 50 | 300 |
| Example | | | | | |
| 2-1 | | | | | |
| Preparation | 100 | 100 | 30 | 70 | 300 |
| Example | | | | | |
| 2-2 | | | | | |
| Preparation | 100 | 140 | 30 | 30 | 300 |
| Example | | | | | |
| 2-3 | | | | | |
| Preparation | 100 | 60 | 70 | 70 | 300 |
| Example | | | | | |
| 2-4 | | | | | |

### Comparative Preparation Example 2. Preparation of Comparative Conditions Heat-Treated Product

A heat-treated product was prepared under the conditions of Preparation Example 2, but the content of each component was prepared under the conditions of Table 5 below.

TABLE 5

| | Cucumis | | | | |
|---|---|---|---|---|---|
| | melo | Sweet | Capsosiphon | | |
| | conomon | Pumpkin | fulvescens | Ecklonia | Water |
| Condition | (g) | (g) | (g) | cava (g) | (g) |
| Comparative | 0 | 250 | 0 | 50 | 300 |
| Preparation | | | | | |
| Example 2-1 | | | | | |
| Comparative | 250 | 0 | 0 | 50 | 300 |
| Preparation | | | | | |
| Example 2-2 | | | | | |
| Comparative | 0 | 0 | 250 | 50 | 300 |
| Preparation | | | | | |
| Example 2-3 | | | | | |
| Comparative | 100 | 100 | 100 | 0 | 300 |
| Preparation | | | | | |
| Example 2-4 | | | | | |

### Example 1 and Comparative Example 1. Preparation of Granular Compositions—I

Under the conditions of Table 6 below, each powder and heat-treated product were mixed, kneaded to make a dough, and granulated using a granulator. At this time, the granulation process is as follows. The dough was molded into granules using a reverse-rotating granulator (Garyeo Industrial Co.), and the molded ones were dried in a drying room at 50° C. for 5 hours so that the moisture content was 3 wt % to 4 wt %, and then granules molded with a diameter of 0.5 mm or more were recovered by sieving.

Among the granule raw materials, the powder of locust adult and silkworm pupa was prepared and used as follows, adult locust adult and silkworm pupa was purchased from an insect breeding farm, steamed for 30 minutes, dried, and then baked in an oven at 180° C. for 3 hours.

TABLE 6

| | Cultured | Preparation | | | |
|---|---|---|---|---|---|
| | mycelium | Example 2-1 | | | |
| | complex of | Heat- | | Locust | Silkworm |
| | Preparation | treated | | adult | pupa |
| | Example 1 | product | Water | powder | powder |
| Condition | Powder (g) | (g) | (g) | (g) | (g) |
| Example 1-1 | 100 | 100 | 0 | 50 | 50 |
| Example 1-2 | 100 | 100 | 0 | 30 | 70 |
| Example 1-3 | 100 | 100 | 0 | 70 | 30 |
| Example 1-4 | 100 | 80 | 0 | 60 | 60 |
| Comparative | 100 | 100 | 0 | 100 | 0 |
| Example 1-1 | | | | | |
| Comparative | 100 | 0 | 100 | 50 | 50 |
| Example 1-2 | | | | | |
| Comparative | 100 | 50 | 50 | 50 | 50 |
| Example 1-3 | | | | | |
| Comparative | 200 | 100 | 0 | 0 | 0 |
| Example 1-4 | | | | | |

### Example 2 and Comparative Example 3. Preparation of Granular Composition—II

Granules were prepared by applying the granule preparing process of Example 1 and Comparative Example 1, but by changing the conditions of the heat-treated product to the conditions of Table 7 below.

TABLE 7

| Condition | Characteristic |
|---|---|
| Example 2-1 | Granules prepared by heat- |
| | treated product of Preparation |
| | Example 2-2 |
| Example 2-2 | Granules prepared by heat- |
| | treated product of Preparation |
| | Example 2-3 |
| Example 2-3 | Granules prepared by heat- |
| | treated product of Preparation |
| | Example 2-4 |
| Comparative | Granules prepared by heat- |
| Example 2-1 | treated product of Comparative |
| | Preparation Example 2-1 |
| Comparative | Granules prepared by heat- |
| Example 2-2 | treated product of Comparative |
| | Preparation Example 2-2 |
| Comparative | Granules prepared by heat- |
| Example 2-3 | treated product of Comparative |
| | Preparation Example 2-3 |
| Comparative | Granules prepared by heat- |
| Example 2-4 | treated product of Comparative |
| | Preparation Example 2-4 |

### Experimental Example 2. Check the Degree of Granulation

Table 8 below shows whether granules are well produced according to the preparation conditions of the heat-treated product. It was determined that the granulation was good when it was 90% or more to maintain an intact form after the final preparation, and the condition was described when no granulation molding was performed or more than 5% of broken granulation was found during the drying process even after molding.

TABLE 8

| Condition | State |
|---|---|
| Example 1-1 | Good |
| Example 1-2 | Good |
| Example 1-3 | Good |
| Example 1-4 | Good |
| Example 2-1 | Good |
| Example 2-2 | Good |
| Example 2-3 | Good |
| Comparative | Poor granulation using a granulator |
| Example 1-1 | |
| Comparative | Poor granulation using a granulator |
| Example 1-2 | |
| Comparative | Poor granulation using a granulator |
| Example 1-3 | |
| Comparative | Poor granulation using a granulator |
| Example 1-4 | |
| Comparative | Crumbling during drying process after |
| Example 2-1 | granule preparing |
| Comparative | Crumbling during drying process after |
| Example 2-2 | granule preparing |
| Comparative | Crumbling during drying process after |
| Example 2-3 | granule preparing |
| Comparative | Crumbling during drying process after |
| Example 2-4 | granule preparing |

As a result, it can be confirmed that the granule formulation is completely prepared in Examples 1 and 2, and the conditions of Comparative Examples 1 and 2 were not well kneaded in the granulator, resulting in poor granulation formation or crumbling while drying the produced granules.

Through this, it can be seen that the content of each raw material sample, which is a raw material for granules, or the conditions under which the heat-treated product was prepared in particular among the raw material samples are important.

### Experimental Example 3. Check the Blood Sugar Lowering Effect by Taking Granules

Five people in their 40s and 60s who had more than 10 days of experience during 1 month measuring symptoms with an empty blood sugar of 110 to 130 mg/dl (pre-diabetes state, diabetes must exceed 130 mg/dl) were allowed to take the compositions of Examples 1 and 2 respectively for 1 month and the period during which the fasting blood sugar increased to 110 mg/dl or more was measured during the dosing period. Although they were not provided with a separate dietary table, they were instructed to take 5 g of each granule within 30 minutes after each meal. The measured values are shown in Table 9 below, and the number of occurrences of hyperglycemia symptoms for each group was recorded as an average value. The decimal point of the average value is rounded off. At this time, the group ingesting the cultured mycelium complex powder of Preparation Example 1 was also set as the comparative group.

As a result of the confirmation, it can be seen that the number of times of hyperglycemia was significantly reduced for 1 month in all the granules taking groups of Examples 1 and 2. In particular, the effect was similar to that of the complex cultured mushroom mycelium powder of Preparation Example 1, which is a result of proving that the continuous intake is more important than the intake amount of the complex cultured mushroom.

TABLE 9

| | Number of times of |
|---|---|
| | hyperglycemia in 1 |
| Condition | month while taking |
| Example 1-1 | 2 |
| Example 1-2 | 3 |
| Example 1-3 | 2 |
| Example 1-4 | 3 |
| Example 2-1 | 2 |
| Example 2-2 | 1 |
| Example 2-3 | 3 |
| Preparation Example 1 | 2 |

### Experimental Example 4. Sensory Evaluation

Through the results of the previous blood sugar drop effect, the following sensory evaluation confirmed whether granular formulations are easy to take and whether they can be applied as health supplements that can relieve hunger, which usually occurs in people with high blood sugar symptoms. In this case, among the samples that were tried for granulation formulation, those that were not granulated well were mixed with each raw material sample, dried, and then powdered to be taken.

To this end, adults in their 20s and 60s, men and women of all ages were divided into two groups, and 5 g of the powder or granules were taken within 30 minutes after every meal in 20 people for 3 days. Take it on an empty stomach to relieve hunger or check the taste preference for 1 to 5 points (1 point: Very bad to 5 points: Very good) was scored. In addition, in this case, the cultured mycelium complex powder of Preparation Example 1 was presented as a comparative group.

TABLE 10

| Condition | Relieve hunger | Taste preference |
|---|---|---|
| Example 1-1 | 4.3 | 4.1 |
| Example 1-2 | 4.2 | 4.5 |
| Example 1-3 | 4.3 | 4.2 |
| Example 1-4 | 4.1 | 4.1 |
| Example 2-1 | 4.2 | 4.4 |
| Example 2-2 | 3.9 | 4.2 |
| Example 2-3 | 4.2 | 3.8 |
| Comparative | 2.4 | 2.9 |
| Example 1-1 | | |
| Comparative | 2.5 | 2.8 |
| Example 1-2 | | |
| Comparative | 2.9 | 2.1 |
| Example 1-3 | | |
| Comparative | 2.1 | |
| Example 1-4 | | |
| Preparation | 2.3 | |
| Example 1 | | |

As a result, it can be confirmed that the granule formulations of Examples 1 and 2 are helpful in relieving hunger and have good taste, so they are good products for use as health supplements. In particular, it can be seen that the cultured mycelium complex itself is significantly superior to the powder, and it is also confirmed that the raw material of the granules and the preparing conditions of the heat-treated product affect both the feeling of hunger and the taste of the granule formulation.

1. A method of preparing a blood sugar control composition containing a cultured mushroom mycelium complex, the method comprising:
(Step 1) inoculating the fruiting body tissues of three types of mushrooms including Innotus obliquus, Phellinus linteus, and Ganoderma lucidum into potato dextrose agars (PDA), respectively and culturing the mycelium of each of the mushrooms;
(Step 2) inoculating a mycelium complex including the mycelium of each of the Innotus obliquus, Phellinus linteus, and Ganoderma lucidum cultured in Step 1 into potato dextrose broth (PDB);
(Step 3) culturing the potato dextrose broth (PDB) inoculated with the mycelium of each of the three types of mushrooms for 4 to 6 weeks;
(Step 4) inoculating the mycelium obtained through Step 3 into a rice barley medium; and
(Step 5) obtaining a complex culture mycelium by culturing the mycelium inoculated into the rice barley medium for 4 to 7 weeks.

2. A composition for prevention or treatment of diabetes, the composition comprising a mushroom complex culture mycelium prepared by the method of claim 1.

3. A healthy functional food for prevention or treatment of diabetes, the food comprising a mushroom complex culture mycelium prepared by the method of claim 1.

4. A granule composition for controlling blood sugar and dietary, the composition comprising heat-treated products of Cucumis melo conomon, sweet pumpkin, Capsosiphon fulvescens, and Ecklonia cava in powder of a mushroom complex culture mycelium prepared by the method of claim 1.

5. A composition for prevention or treatment of diabetes, the composition comprising the granule composition of claim 4.

6. A healthy functional food for prevention or treatment of diabetes, the food comprising the granule composition of claim 4.

7. A method for preventing or treating diabetes, comprising administering the composition of claim 2 to a subject in need thereof.
